(19) United States
(12) Patent Application Publication
(10) Pub. No.: US 2014/0135874 A1
(43) Pub. Date: May 15, 2014

(54) DEVICE FOR WOUND TREATMENT THROUGH PHOTOBIOMODULATION

(71) Applicants: MONICA DEAN, Chattanooga, TN (US); MATTHEW DEAN, Chattanooga, TN (US)

(72) Inventors: MONICA DEAN, Chattanooga, TN (US); MATTHEW DEAN, Chattanooga, TN (US)

(21) Appl. No.: 14/078,452

(22) Filed: Nov. 12, 2013

Related U.S. Application Data
(60) Provisional application No. 61/725,057, filed on Nov. 12, 2012.

(51) Int. Cl. A61N 5/06

(52) U.S. Cl. CPC A61N 5/0613
USPC 607/90

(57) ABSTRACT

A bandage, band-aid, patch, or similar covering with a plurality of LEDs or pico-LEDs arranged on the interior of the device in proximity to the skin of the user when applied. The device may be of any size or shape, including an oval, rectangle, square, or typical “band-aid.” Power is supplied by one or more batteries or other power source, which may include solar power or a power source powered by contact with the skin of the user. The battery may be a flexible thin film battery. Hydrogel may be used for cooling and moisturizing, which promotes cellular and vascular recovery, and also may serve as an adhesive.

This application claims benefit of and priority to U.S. Provisional Application No. 61/725,057, filed Nov. 12, 2012, by Monica Dean, et al., and is entitled to that filing date for priority. The specification, figures and complete disclosure of U.S. Provisional Application No. 61/725,057 are incorporated herein by specific reference for all purposes.

### FIELD OF INVENTION

This invention relates to a device and method for the treatment of wounds and injuries through photobiomodulation.

### BACKGROUND OF THE INVENTION

The use of light therapies in human and veterinary medicine is well known, including photodynamic therapy (the application of light to clinical therapeutics) and photobiomodulation (the application of light to promote cell growth and recovery from injury). In general, photobiomodulation uses light emitting diodes (LEDs) or low energy lasers to emit light in various ranges. For example, the use of visible light modulation for treatment of various diseases is discussed in Dunning, et al., US 2012/0065709 (U.S. application Ser. No. 13/076,114), which is incorporated herein by specific reference in its entirety for all purposes.

At present, the devices used for photobiomodulation are cumbersome and expensive, require external power sources, or are inefficient. Accordingly, what is needed is an improved, portable, self-contained photobiomodulation device.

### SUMMARY OF THE INVENTION

In various exemplary embodiments, the device of the present invention comprises a bandage, band-aid, patch, or similar covering with a plurality of LEDs or pico-LEDs (or similar light sources, such as low-level lasers) arranged on the interior of the device in proximity to the skin of the user when applied. The device may be of any size or shape, including an oval, rectangle, square, or typical “band-aid.” Pico-LEDs are the smallest form of LEDs manufactured today, and contain no glass or harmful chemicals.

The device also contains one or more batteries or other power source (which may include solar power or a power source powered by contact with the skin of the user). In one embodiment, the battery is a flexible thin film battery. Silver may be used for the conductor and FCB (silver has the advantage of also being a microbial agent with use in health care, including pediatric care).

The device also may comprise hydrogel (comprising a mixture of water and alcohol) for cooling and moisturizing, which promotes cellular and vascular recovery. The hydrogel provides comfort to the area to which the device is applied, and also may serve as an adhesive. Alternatively, other forms of adhesive known in the art may be used to adhere the device to the skin of the user in a particular location.

This device provides a significant advantage in that the LEDs are placed directly on (i.e., in direct contact with) or in close proximity to the skin/epidermis of the user, in contrast to prior art devices. The proximity (i.e., less than 1 mm) of the light sources to the epidermis allows a broader range of treatment below the epidermis. The power density decreases when the distance between the LED and the skin surface is increased.

Extreme super luminous diodes (ESLD) may be used. The arrangement and placement of the LEDs on the interior of the device may vary. Likewise, the intensity and color of the LEDs may vary. This arrangement may be constant, or the different colors and intensity may vary over time, randomly or according to an established program. Colors may alternate, or strobe, or some combination thereof. In one exemplary embodiment, the device may include a microchip processor or controller to control the variations in color or intensity.

### DESCRIPTION OF THE DRAWINGS

FIGS. 1-3 show views of an oval-shaped device in accordance with an embodiment of the present invention.

FIGS. 4-6 show views of a square-shaped device in accordance with an embodiment of the present invention.

FIGS. 7 and 8 show interior and exterior views of alternative embodiments of the present invention.

FIG. 9 shows a schematic diagram of a single-LED embodiment of the present invention.

### DETAILED DESCRIPTION OF EXEMPLARY EMBODIMENTS

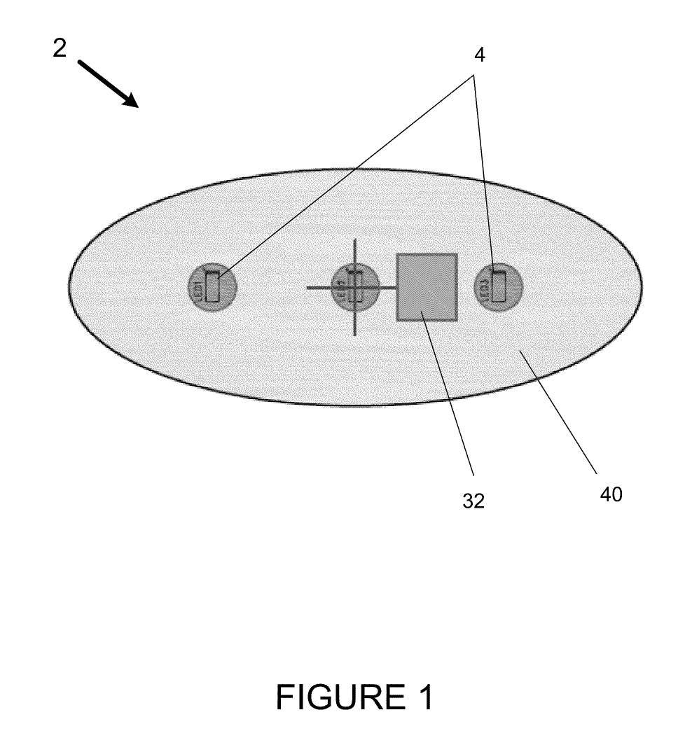

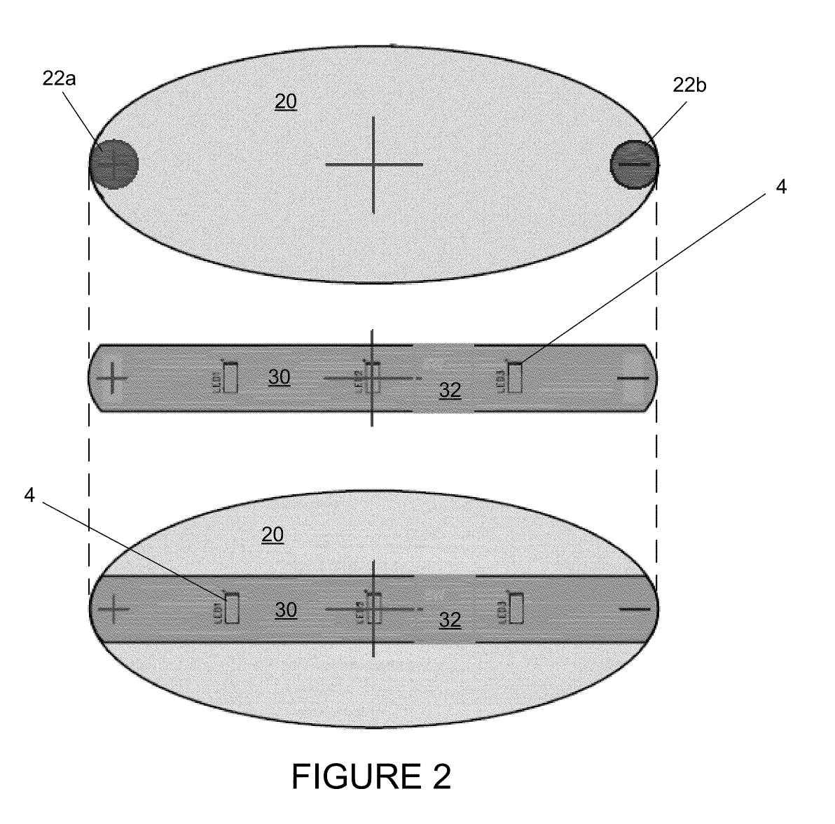

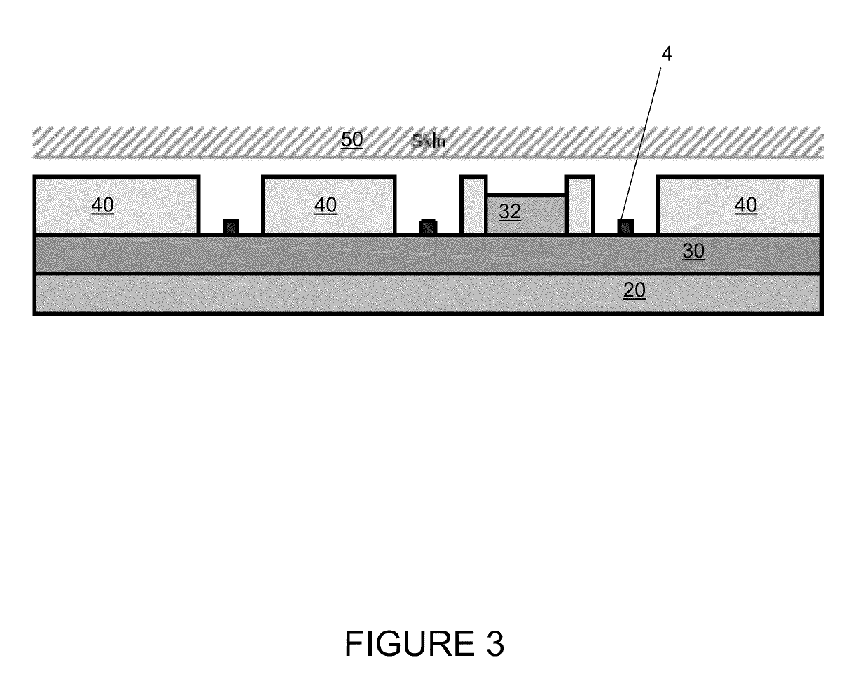

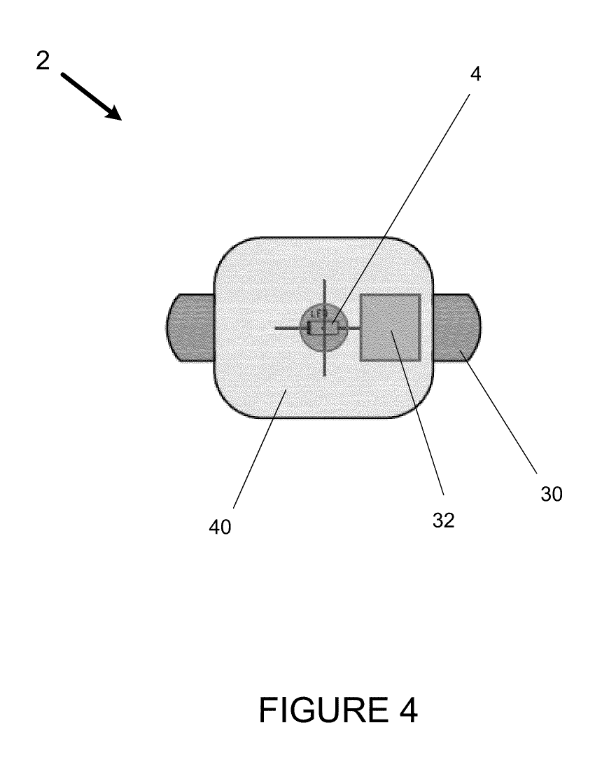

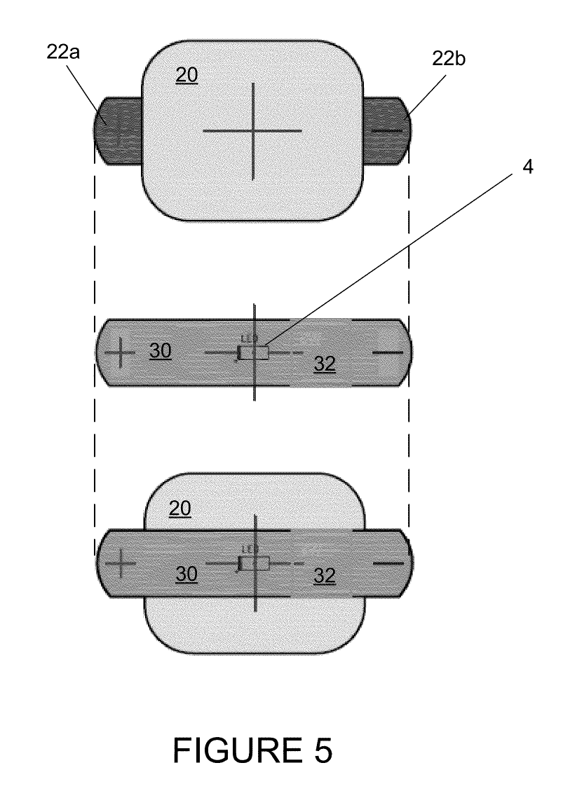

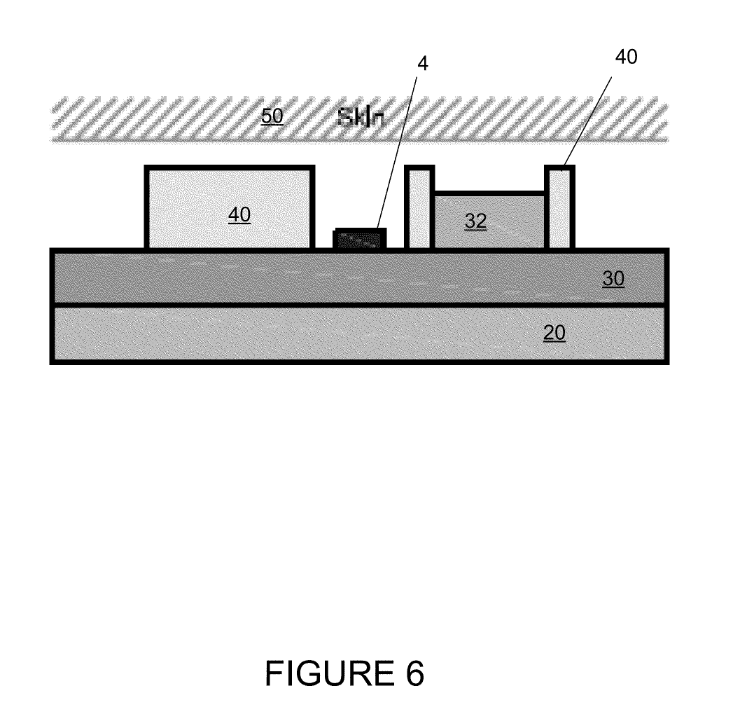

In various exemplary embodiments, as seen in FIG. 1, the device of the present invention comprises a bandage, band-aid, patch, or similar covering 2 with a plurality of LEDs or pico-LEDs 4 (or similar light sources, such as low-level lasers) arranged on the interior of the device in proximity to the skin of the user when applied. The device may be of any size or shape, including an oval, rectangle, square, or typical “band-aid,” as seen FIGS. 1-6. In one embodiment, the device may be 6′×6′ in size. In another embodiment, the device may be 2″×2″ in size. Pico-LEDs are the smallest form of LEDs manufactured today, and contain no glass or harmful chemicals. The LEDs also may be printed, or provided in liquid form. The device also contains one or more batteries or other power source (which may include a solar power cell or a power source powered by contact with the skin of the user).

In several embodiments, the present invention is multi-layered. As seen in FIGS. 1-6, the present device may comprise an exterior covering layer, a battery layer, a flexible circuit layer, and an interior layer (adjacent to the skin 50). The exterior covering layer may contain the battery 22 or batteries or similar power source, and may thus comprise a single layer 20. The flexible circuit layer 30 may comprise a flexible circuit strip with the LEDs or pico-LEDs 4 mounted or printed thereon, and is electrically connected to the battery. A switch 32 on the flexible circuit layer may be used to turn the device power off and on. In one embodiment, the battery is a flexible thin film battery with positive and negative terminals 22a, b. Silver may be used for the conductor and FCB (silver has the advantage of also being a microbial agent with use in health care, including pediatric care).

The flexible circuit layer may comprise the interior layer. Alternatively, a thin covering may be affixed to the flexible circuit layer on the side with the LEDs or pico-LEDs. Openings may be provided for the LEDs or pico-LEDs, and may also be provided for the switch. In the embodiment shown, the interior layer comprises a layer of hydrogel 40. The hydrogel, which comprises a mixture of water and alcohol, provides cooling and moisturizing effects, thereby promoting cellular and vascular recovery. The hydrogel provides comfort to the area to which the device is applied, and also may serve as an adhesive. Alternatively, other forms of adhesive (with or without hydrogel) as known in the art may be used to adhere the device to the skin of the user in a particular location. The hydrogel or adhesive also may contain antimicrobial agents, cosmetic agents, or other medicaments.

In one exemplary embodiment, the hydrogel or adhesive is applied over all or substantially all of the interior side of the device (i.e., adjacent to the skin 50), except for areas around the LEDs or pico-LEDs. This permits the LEDs or pico-LEDs to come into contact with (i.e., be placed directly on) the skin or epidermis, or be in very close proximity (i.e., 1 mm or less) to the skin. This is a significant advantage over prior art devices. The proximity (i.e., 1 mm or less in distance) of the light sources to the epidermis allows a broader range of treatment below the epidermis. The power density decreases when the distance between the LED and the skin surface is increased. In close proximity, however, the effective radius of coverage on the epidermis from a single LED is smaller, albeit deeper, and thus additional LEDs or pico-LEDs may be used, with closer spacing. When treated with photobiomodulation therapy, cellular membranes continued to progress for three days after treatment.

In one exemplary embodiment, extreme super luminous diodes (ESLD) may be used. The arrangement and placement of the LEDs on the interior of the device also may vary. Likewise, the intensity and color of the LEDs may vary. This arrangement may be constant, or the different colors and intensity may vary over time, randomly or according to an established program. Colors may alternate, or strobe, or some combination thereof. In one exemplary embodiment, the device may include a microchip processor or controller 54 to control the variations in color or intensity.

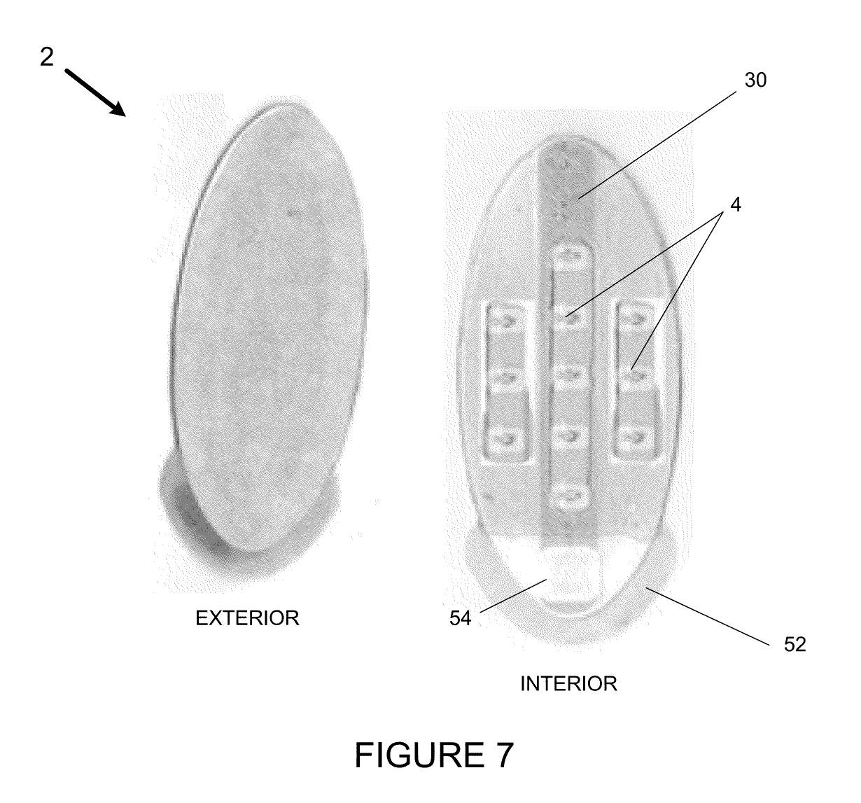

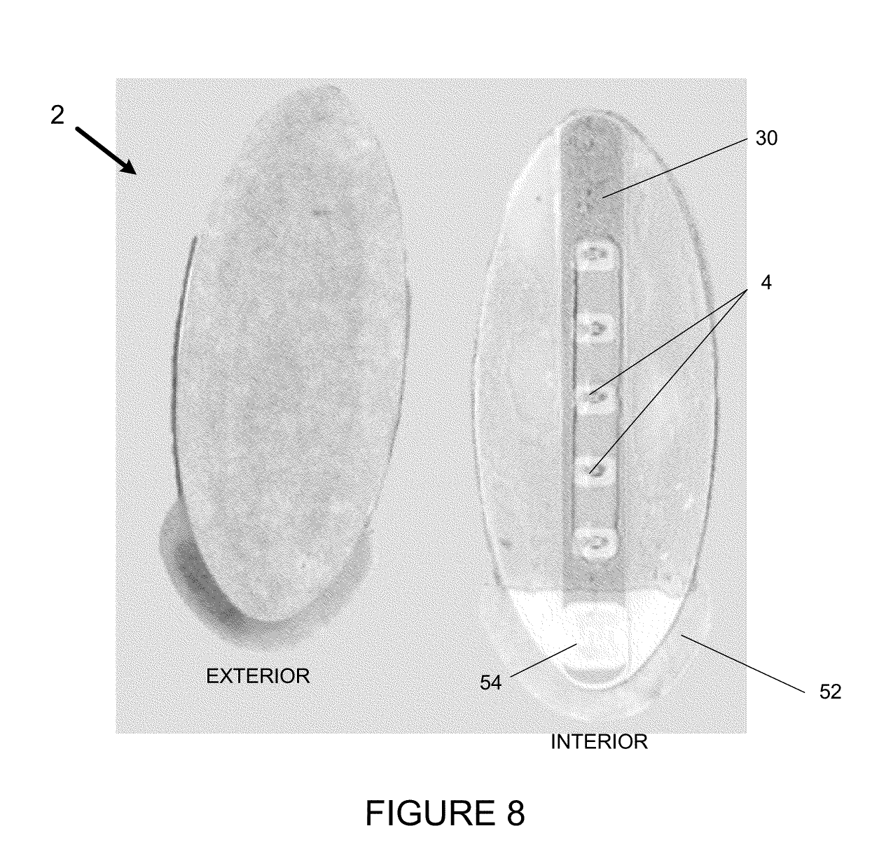

FIG. 7 shows the exterior and interior of another embodiment of the present invention with three strips of LEDs. A flap or tab 52 may be provided to assist with placement and handling of the device (i.e., for application and removal). A flap or pull tab (including a disposable pull tab) between the positive and negative connections also may be used as a power-on switch to allow the battery or power source to initially operate. FIG. 8 shows a similar embodiment, but with a single strip of five LEDs.

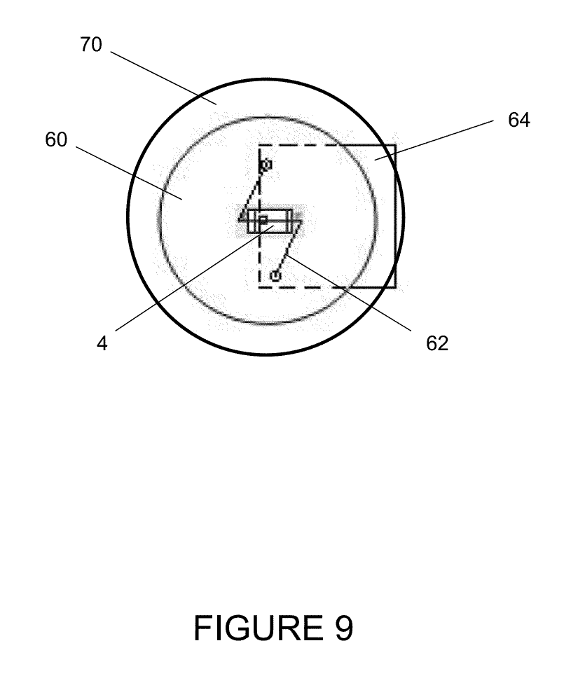

In yet another embodiment, each LED or pico-LED may be a self-contained powered unit, as seen in FIG. 9. The LED or pico-LED 4 is mounted on or with a battery 60 and is electrically connected thereto by a pair of connectors 62. The battery and pico-LED are mounted on a backing, which may be cloth, plastic, rubber, or other synthetic or similar material 70. A disposable pull-tab 64 initially provides separation between the positive and negative connections, and is pulled to complete the circuit and cause the pico-LED to emit light. Hydrogel (or similar adhesive) may be placed on the side of the unit proximate to the skin (in a similar manner as described above), and a peel-off backing layer may be used in a pre-packaged embodiment of the device. In one embodiment, the power supply is sufficient to cause the emission of light for at least 20 minutes.

In one exemplary embodiment, the pico-LED is approximately 1 mm long, 0.6 mm wide, and 0.2 mm thick. Examples include, but are not limited to, the Rohm SML series of pico-LEDs, which come in a variety of colors. In general, LEDs may be provided in various intensities through the ultraviolet, visible, and near-infrared ranges (e.g., 300 nm to 1000 nm).

In several embodiments, the device may use the following colors and intensities of LED light sources:

Blue @470 nm; 550,000 mcd (candelas) or 89 W/cm2

Green @525 nm; 1,300,000 mcd or 27 mW/cm2

Yellow @590 nm; 550,000 mcd

Red @640 nm; 1,000,000 mcd

Blue light is used as a bacteria killer or antimicrobial. Red light is known to promote the repair and healing of cells and cell membranes, and rejuvenate healthy cell reproduction. Yellow light is known to reduce redness, drain fluid, and reduce pain.

Various embodiments of the device may be used for a variety of applications, including but not limited to the following:

| Rapid | | | | |
|---|---|---|---|---|
| Wound | Derm. | Cosmetic | | Misc. |
| Healing | application | Application | Hospital Aid | application |
| Military | Psoriasis | Acne | Specialties | Arthritis |
| combat | Rosacea | Keratosis | Burn Surgery | Pain |
| Diabetic | Eczema | anti-ageing | Cardiovascular | Sports injury |
| Ulcers | | Redness | Emergency | Hair growth |
| Post | | Scaring | Medicine | Onychomycosis |
| ocular | | Cell | General | (nail |
| surgery, | | turnover | Surgery | fungus) |
| Elderly | | Breast | Infectious | HHV-1-2 |
| bed | | Aug. | Disease | (herpes) |
| sores | | Tummy | Thoracic | Photodynamic |
| Children | | tuck | Surgery | therapy |
| bandages | | Cosmetic | Transplant | Pink eye |
| | | surgery | Surgery | Athletes foot |
| | | Teeth | Trauma & | |
| | | whitening | Acute | |
| | | | Surgery | |
| | | | Neonatal | |
| | | | phototherapy | |
| | | | blankets | |
| | | | Catheter BSI | |

In one exemplary embodiment, for example, a device in accordance with the present invention is used to treat a catheter port site. For this application, the LEDs may be blue (@460-500 nm, and 550,000 mcd), which has antimicrobial applications. This embodiment treats bacteria below the surface of the skin, blood clotting, and reduces swelling, redness and soreness. The device stops blood stream infections by killing any bacteria before it enters the blood stream. The frequency range may be varied, depending on whether the device is being used to treat an existing infection, or for general bacterial control.

In another exemplary embodiment, the LEDs may be red (as described above) for a wound healing application. Red light promotes healthy tissue growth, and treatment will accelerate wound healing by a factor of three over traditional treatments. Red light also may have antimicrobial effects.

As noted above, the present invention has the advantage of combining light colors and strobing in a device in direct proximity to the skin, providing an increased effectiveness in photobiomodulation treatment. The light reaches deep into the subcutaneous tissue for deep wound treatment, blood clotting, and cellular reconstruction. The combination of colors and stroking effects may be matched to specific treatment solutions, and each skin condition should be treated accordingly to its symptoms. For example, if the skin disorder is easily agitated by heat or physical activity, the treatment would not include red light or strobing, as this may further agitate the skin disorder, causing pain or discomfort.

Another significant advantage is that the present apparatus is designed to be disposable and less expensive to produce. The apparatus can be easily obtained and applied by users, and disposed of after the therapy is completed with that particular device.

Thus, it should be understood that the embodiments and examples described herein have been chosen and described in order to best illustrate the principles of the invention and its practical applications to thereby enable one of ordinary skill in the art to best utilize the invention in various embodiments and with various modifications as are suited for particular uses contemplated. Even though specific embodiments of this invention have been described, they are not to be taken as exhaustive. There are several variations that will be apparent to those skilled in the art.

What is claimed is:

1. A device, comprising:
a backing layer with an exterior side and an interior side;
one or more light sources;
a flexible circuit strip or board affixed to the interior side of the backing layer, wherein the light sources are mounted on said flexible circuit strip or board;
a power source providing electrical power to said light sources; and
an adhesive layer applied to the interior side of said backing layer.

2. The device of claim 1, wherein said light sources are LEDs.

3. The device of claim 1, wherein said light sources are pico-LEDs.

4. The device of claim 1, wherein said power source is a thin film battery.

5. The device of claim 1, wherein said power source is a solar cell.

6. The device of claim 1, wherein there is a single light source.

7. The device of claim 1, wherein the light sources are arranged in one or more lines.

8. The device of claim 1, wherein the adhesive layer is hydrogel.

9. The device of claim 1, wherein the adhesive layer is not applied over the light sources.

10. The device of claim 1, wherein the device is adapted to be placed on the human epidermis.

11. The device of claim 10, wherein the light sources are no more than 1 mm distant from the epidermis when the device is placed on the human epidermis.

12. The device of claim 1, wherein the light sources are identical in color and intensity.

13. The device of claim 1, wherein the light sources vary in color and intensity.

14. The device of claim 1, wherein one or more light sources are strobing.

15. The device of claim 1, further comprising a microchip processor or controller mounted or embedded in said flexible circuit strip or board.

16. The device of claim 15, wherein said microchip processor or controller is programmed to control the color and intensity of the light sources.

17. The device of claim 1, further comprising a switch to turn the light sources on or off.
